(19) United States
(12) Patent Application Publication
(10) Pub. No.: US 2013/0274641 A1
(43) Pub. Date: Oct. 17, 2013

(54) Achilles tendon protector

(71) Applicant: Glenn Douglas Dolfi, JR., Galena, OH (US)

(72) Inventor: Glenn Douglas Dolfi, JR., Galena, OH (US)

(21) Appl. No.: 13/863,518

(22) Filed: Apr. 16, 2013

Related U.S. Application Data
(60) Provisional application No. 61/624,660, filed on Apr. 16, 2012.

(51) Int. Cl. A61F 5/01

(52) U.S. Cl. CPC A61F 5/0102
USPC 602/23

(57) ABSTRACT

A guard for a rear ankle area of a wearer. A top portion is adapted to rest against a lower calf portion of the wearer. A middle portion is integrally formed to the top portion and extends therefrom curving outward to terminate at a bottom portion, wherein, in combination, the top portion, the middle portion and the bottom portion are adapted to align with and shield a substantial portion of an Achilles tendon of the wearer. The bottom portion has a bottom lateral end connected thereto extending radially therefrom to form a lateral cup, and the bottom portion has a bottom medial end connected thereto and extending therefrom radially on an opposite side of the bottom portion in an opposite direction from the bottom lateral end to form a medial cup for placement, respectively, over a lateral malleolus and medial malleolus of the wearer.

### CROSS-REFERENCE TO RELATED APPLICATIONS

The instant application claims benefit of provisional application Ser. No. 61/624,660 filed Apr. 16, 2012, the contents of which are herein incorporated by reference.

### BACKGROUND

1. Field of the Invention:

The instant invention relates to guards to protect a wearer's leg and ankle areas, particularly during sporting events. Specifically, the instant invention is a guard for a user's Achilles tendon and rear ankle area.

2. Description of the Related Art:

Most leg guards, for instance for use for American Soccer, are designed to be worn at the shin to protect the tibia of the player, which contact inevitably occurs as players frequently strike each others' legs inadvertently. The shin guards therefore reside at the front of the leg above the wearer's ankle over the wearer's shin to absorb impacts. Some shin guards contain extra padding for disposition along the lower sides of the ankle, for instance over the lateral malleolus and medial malleolus. For example U.S. Pat. No. 6,298,484 to Beckman teaches a protective guard for soccer players which includes a lower padded region below a shin guard for disposition around the wearer's ankle. Inherent in either design is that the rear of the leg and ankle of the user is still substantially exposed.

Attempts have been made to provide guards which better protect the ankle regions, which can be used with or in place of traditional shin guards. U.S. Design Pat. No. D593,715 to Scheffer shows an ankle protector for example. Notably, however, is that in most cases ankle wraps are not anatomically correct because the portions which are disposed over the lateral malleolus and opposing medial malleolus are aligned, when in fact the medial malleolus is higher above the ankle than the lateral, or outside, malleolus. Because of this the same amount of padding or impact protection is not provided to one of these bone portions and the “feel” of the guard is disproportionate.

In an effort to alleviate rear leg impact concerns, protective guards have been taught which are designed to encompass the entire lower leg, including the calf, ankle, and Achilles tendon area. For example, U.S. Pat. No. 6,305,031 to White shows a protective appliance to be worn completely over the lower leg which includes rigid plates adapted to conform to the wearer's leg. The result of any design which entirely encompasses the calf muscle and rear of the ankle unduly limits the mobility of the wearer during the sporting event however. At a minimum, even if freedom of movement is retained, the wearer can still feel the nagging presence of a guard. Furthermore, excessive material increases the weight of the guard and additionally results in an increase in cost which causes a potential user to forego the benefit of additional lower leg protection.

There is a need then for a rear ankle and Achilles tendon guard which addresses the above problems to provide impact protection while cost-effectively not hindering feel and performance.

### SUMMARY

It is the objective of the instant invention to provide an ankle and Achilles tendon guard which is anatomically correct.

It is further the objective of the instant invention to provide a guard which is lightweight and has only minimal amount of material.

It is additionally the objective of the instant invention to provide a guard which does not bother or hinder the wearer.

It is further the objective of the instant invention to provide a guard which can be used in conjunction with traditional shin guards.

Accordingly comprehended is a guard for a rear ankle area of a wearer comprising a top portion adapted to rest against a lower calf portion of the wearer, the top portion having a top lateral end, a top medial end, and a topmost edge. A middle portion is integrally formed to the top portion and extends therefrom curving outward to terminate at a bottom portion distal to the top portion, wherein, in combination, the top portion, the middle portion and the bottom portion are adapted to align with and shield a substantial portion of an Achilles tendon of the wearer. The bottom portion has a bottom lateral end connected thereto extending radially therefrom to form a lateral cup, and the bottom portion has a bottom medial end connected thereto and extending therefrom radially on an opposite side of the bottom portion in an opposite direction from the bottom lateral end to form a medial cup. The medial cup has a medial center axis offset from a lateral center axis of the lateral cup, wherein the lateral cup is adapted to be positioned over a lateral malleolus of the wearer and the medial cup is adapted to be positioned over a medial malleolus of the wearer. Formed therefore is a unit, protecting, as much as possible, the lower aspect of the leg, in high impact sports where these impacts regularly occur.

### BRIEF DESCRIPTION OF THE DRAWINGS

FIG. 1 shows a left-side perspective view of the instant Achilles guard in use on a wearer's leg.

FIG. 2 shows a rear elevation view thereof.

FIG. 3 shows a front elevation view thereof.

FIG. 4 shows a right-side elevation view thereof.

### DETAILED DESCRIPTION OF THE PREFERRED EMBODIMENT

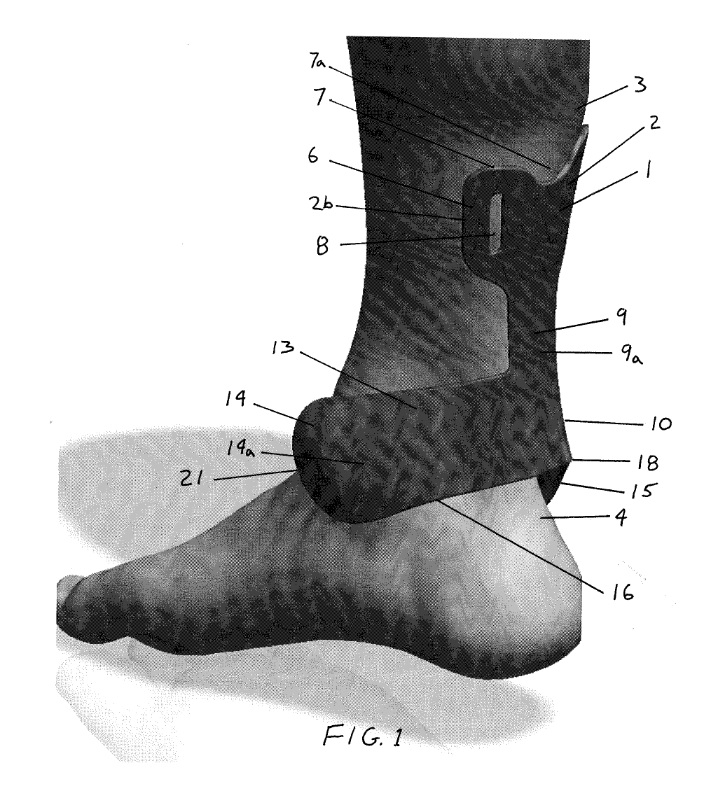

With reference then to FIGS. 1-4, shown is the instant guard 1. “Guard” as used herein means a protective shield for the rear ankle area which for purposes of this embodiment is meant to include a human's (wearer) Achilles tendon 4, lower calf 3, and both sides of the ankle, namely the lateral malleolus and medial malleolus and surrounding inside (medial) and outside (lateral) areas above the heel. The guard 1 is preferably made of lightweight, cost effective hard plastic, carbon fiber, or other material designed to absorb and deflect impact. The unit as a whole will disperse the energy from an impact throughout. The aspect of the guard 1 in contact with the wearer/player's skin will have a thin gel and foam layer to form to the player's specific leg characteristics. It should be understood that the size and dimensions of the guard 1 may vary and can be made in multiple sizes and/or be customized for fit and function based on human anatomy. It should be further understood for the preferred embodiment that due to the asymmetric nature of the human ankle anatomy, e.g. the lateral malleolus is not directly opposite the medial malleolus, there would typically be two different guards, one for the left leg and one for the right leg, as further described and which is herein a critical improvement.

Accordingly, the guard 1 comprehends a top portion 2 adapted to rest against lower calf portion 3 of a human wearer. Since it is known the Achilles tendon 4 of a human connects the heel bone to the two calf muscles, lower calf portion 3 means the area proximate to where the Achilles tendon 4 connects to the calf. Top portion 2 is preferably Y-shaped having a top lateral end 5, a top medial end 6, and a topmost edge 7. For comfort, topmost edge 7 can include an edge cut-out 7a defined therein which more anatomically follows the contour of the top of the Achilles tendon 4b as opposed to a straight edge for instance. Moreover, by eliminating some of the semi-rigid material at this location better freedom of movement is enabled during use when the wearer is periodically flexing and un-flexing the calf muscle. To aid in the securement of the guard 1 onto the wearer's rear ankle area, a means for securing the guard to the wearer's rear ankle area can be included, and such means for securement may include one or more strap slits 8 defined within top portion 2 adapted to allow a fastening strap or strip of hook-and-loop fasteners to pass through and around the guard. Such strap slits 8 can be positioned on various locations along the guard 1 (not shown). The strap may optionally come from the shin guard of the wearer, resulting in the wearer's leg being guarded from both front to back, almost in its entirety.

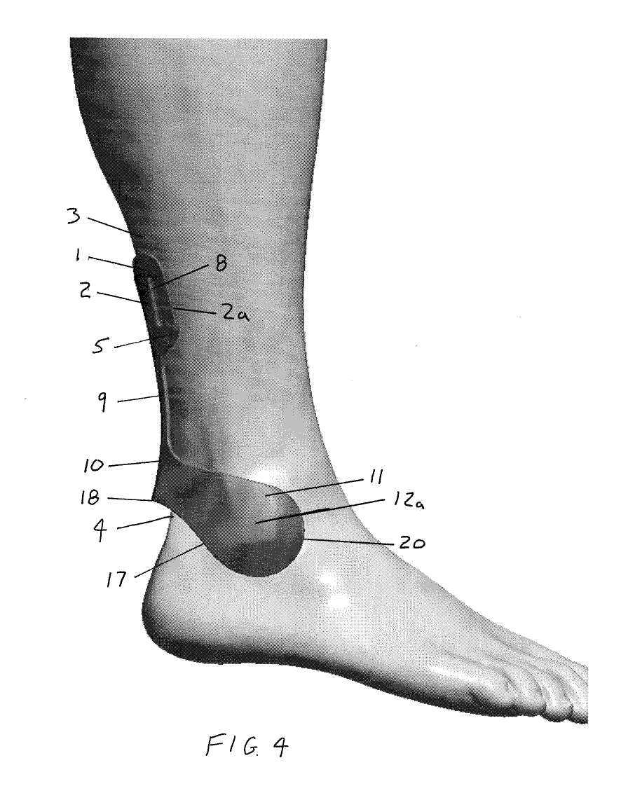

Middle portion 9 is integrally formed to the top portion 2 and extends therefrom (downward when in use). Specifically, top portion 2 includes a top lateral edge 2a and top medial edge 2b which curve downward to form middle portion 9 with middle portion 9 preferably shaped to have a middle width 9a which is slightly greater than the approximate width of the wearer's Achilles tendon 4. For instance, in the preferred embodiment middle width 9a of middle portion 9 is in the range of 1¼ inches to 2% inches, which can vary depending on the “size” of the guard, e.g. adult large, youth medium, etc. By additionally having a middle width 9a which is minimized and less than the width of the top portion 2, material and therefore weight is eliminated to enhance comfort and feel without sacrificing function. Middle portion 9 curves slightly outward as it travels downward away from or distal to top portion 2. “Outward” in this respect means as would be relative to the rear ankle area of the wearer, following the contour of this region (see FIG. 1 or 4 for instance). Thereafter, middle portion 9 terminates at bottom portion 10.

Bottom portion 10 therefore is integral with middle portion 9 and distal to top portion 2. In combination therefore, bottom portion 10, middle portion 9, and top portion 2 are adapted to align with and shield a substantial portion of an Achilles tendon 4 of the wearer. “Substantial” as used here means there is a slight portion of the Achilles tendon 4 which is allowed to remain exposed or unshielded, just above the heel bone, as further described.

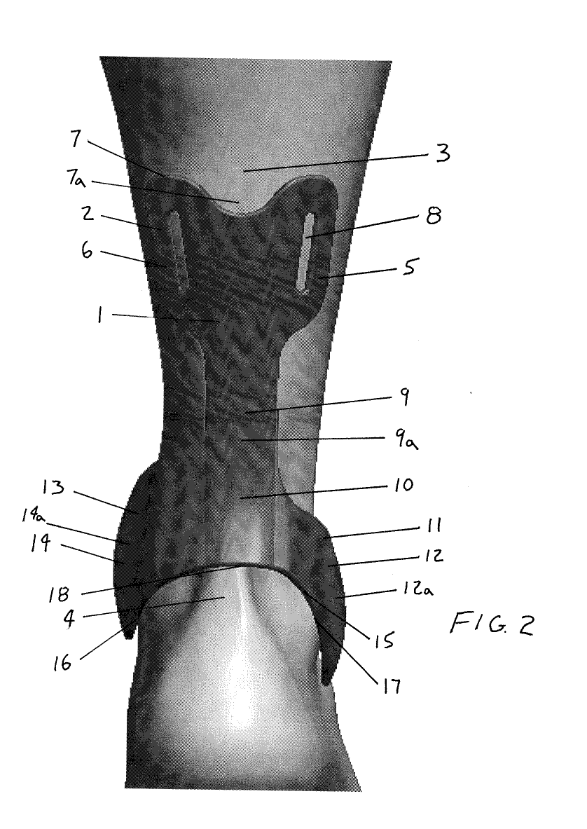

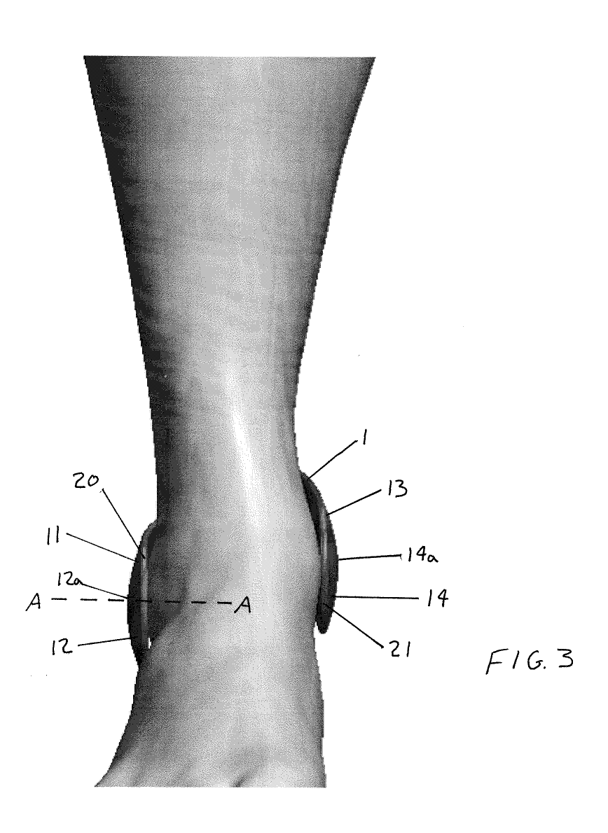

Bottom portion 10 has a bottom lateral end 11 connected to but extending away from the bottom portion 10 and radially therefrom to form a lateral cup 12, “radially” extending in that there is a slight curvature from the bottom portion around to the front edge 20 and forward edge 21 (see FIG. 3 for instance) toward the front of wearer's ankle. Extending radially from an opposite side of the bottom portion 10 in an opposite direction from bottom lateral end 11 is the bottom medial end 13 which forms medial cup 14. As shown then with reference to FIGS. 1 and 4, when viewed from the side in elevation the guard 1 is generally L-shaped. Each “cup” 12, 14 therefore is adapted to reside over and “cup” or shield the respective lateral malleolus and medial malleolus area of the wearer. Of note, and critically, is that to accomplish accurate “cup” placement, the medial cup 14 has a medial center 14a which is axially offset from a lateral center 12a of the lateral cup 12, for instance away from line A-A of FIG. 3. This is in conformity with the anatomy of a human ankle since the lateral malleolus is lower towards the foot than the medial malleolus, hence the asymmetrical nature of the guard and difference in right and left foot variations. As shown with reference to FIG. 2, a height of the bottom medial end 13 along the bottom portion 10 may not be identical to the size of the bottom lateral end 11. There is a tendency to be impacted more on the inside of the ankle area than on the outside, hence in the preferred embodiment the bottom medial end 13 has a larger area than the bottom lateral end 11.

A curved bottommost edge 15 is formed at bottom portion 10 as the medial bottom edge 16 is co-linear with the lateral bottom edge 17. Curved bottommost edge 15 includes an apex 18 formed at a juncture of the lateral bottom edge 17 and the medial bottom edge 16. The apex 18 is adapted to reside adjacent to (just above) a rear upper of a shoe being worn by the wearer. Therefore, in combination, the bottom portion 10, middle portion 9, top portion 2 and the rear upper of the shoe now shield the entirety of the Achilles tendon 4. So notably, as above, the bottom portion 10 is meant to not extend down all the way to the heel so as to not prohibit freedom of movement. In plantar flexion, the guard 1 will rest on the outer aspect of the rear of the shoe, allowing the shoe to take some of the energy of any impact as well. But the entirety of the Achilles tendon 4 is still guarded from impact injury, with the guard 1 further designed to protect and support the user in conjunction with the use of shin guards, to form a unit, protecting, as much as possible, the lower aspect of the leg, in high impact sports where these impacts regularly occur.

I claim:

1. A guard for a rear ankle area of a wearer, comprising:
a top portion adapted to rest against a lower calf of said wearer, said top portion having a top lateral end, a top medial end, and a topmost edge;
a middle portion integrally formed to said top portion and extending therefrom and curving outward to terminate at a bottom portion distal to said top portion, wherein, in combination, said top portion, said middle portion and said bottom portion are adapted to align with and shield a substantial portion of an Achilles tendon of said wearer; and,
said bottom portion having a bottom lateral end connected thereto extending radially therefrom to form a lateral cup, and said bottom portion having a bottom medial end connected thereto and extending therefrom radially on an opposite side of said bottom portion in an opposite direction from said bottom lateral end to form a medial cup, said medial cup having a medial center axis offset from a lateral center axis of said lateral cup, wherein said lateral cup is adapted to be positioned over a lateral malleolus of said wearer and said medial cup is adapted to be positioned over a medial malleolus of said wearer.

2. The guard of claim 1, wherein said middle portion has a middle width of less than a distance between a top lateral end edge and a top medial end edge such that said top portion curves down into said middle portion.

3. The guard of claim 2, wherein said middle width is in the range of 1¼ inches and 2¼ inches.

4. The guard of claim 1, further comprising an edge cut-out defined within said topmost edge.

5. The guard of claim 1, wherein a medial bottom edge is collinear with a lateral bottom edge to form a curved bottommost edge.

6. The guard of claim 5, wherein an apex is formed along said curved bottommost edge at a juncture of said lateral bottom edge and said medial bottom edge.

7. The guard of claim 6, wherein said apex is adapted to reside adjacent to a rear upper of footwear such that, in combination, said bottom portion and said rear upper of said footwear shield the entirety of said Achilles tendon.

8. The guard of claim 1, wherein a height of said medial lower end along said bottom portion is greater than a height of said lateral lower end.

9. The guard of claim 1, further comprising one or more strap slits defined within said top portion.

10. A guard for a rear ankle area of a wearer, comprising:
a Y-shaped top portion having a top lateral end, a top medial end, and a topmost edge;
a middle portion integrally formed to said top portion and extending therefrom and curving outward to terminate at a bottom portion distal to said top portion; and,
said bottom portion comprising a bottom lateral end connected thereto extending radially therefrom to form a lateral cup, and said bottom portion having a bottom medial end connected thereto and extending therefrom radially on an opposite side of said bottom portion in an opposite direction from said bottom lateral end to form a medial cup, wherein a bottom half of said guard is shaped generally of an inverted Y.

11. The guard of claim 10, wherein said middle portion has a middle width of less than a distance between a top lateral end edge and a top medial end edge such that said top portion curves down into said middle portion.

12. The guard of claim 10, further comprising an edge cut-out defined within said topmost edge.

13. The guard of claim 10, wherein a medial bottom edge is collinear with a lateral bottom edge to form a curved bottommost edge.

14. The guard of claim 13, wherein an apex is formed along said curved bottommost edge at a juncture of said lateral bottom edge and said medial bottom edge.

15. The guard of claim 10, wherein a height of said medial lower end along said bottom portion is greater than a height of said lateral lower end.

16. The guard of claim 10, further comprising one or more strap slits defined within said top portion.
